(19) United States
(12) Patent Application Publication
(10) Pub. No.: US 2017/0361026 A1
(43) Pub. Date: Dec. 21, 2017

(54) Syringe cover for Obscuring a Patient's Observation of Needle Insertions

(71) Applicants: Reem Eldawud, Morgantown, WV (US); Dina Eldawud, Morgantown, WV (US); Dauod Eldawud, Morgantown, WV (US)

(72) Inventors: Reem Eldawud, Morgantown, WV (US); Dina Eldawud, Morgantown, WV (US); Dauod Eldawud, Morgantown, WV (US)

(21) Appl. No.: 15/183,030

(22) Filed: Jun. 15, 2016

(51) Int. Cl. A61M 5/32

(52) U.S. Cl. CPC A61M 5/32; A61M 2205/59

(57) ABSTRACT

A syringe cover obscures a patient's observation of needle insertions for medical procedures. Obscuring the needle insertion from the patient reduces the psychological fear associated with needles. The syringe cover includes a tubular sleeve, a collapsible sleeve, and a viewport opening. The tubular sleeve covers the fluid reservoir barrel to hide the fluid drawn from or injected into the patient from the patient's view. The collapsible sleeve obscures the needle of the syringe from the patient's view. The collapsible sleeve is pressed against the patient's body when the needle penetrates the patient's skin. The collapsible sleeve deforms as the needle is inserted into the patient's body such that the collapsible sleeve does not obstruct the insertion of the needle. The viewport allows the medical practitioner to view the volume of fluid within the fluid reservoir barrel of the syringe.

### FIELD OF THE INVENTION

The present invention relates generally to a syringe cover. More specifically, the present invention relates to a syringe cover which obscures the insertion of a needle into a patient from the patient's vision.

### BACKGROUND OF THE INVENTION

Needles are commonly used in many healthcare procedures to promote and diagnose the health of the patient. Medical injections are administered for curative care, immunization, and blood transfusions, as well as many other procedures. Through the use of medicinal injections mankind has seen a drop in number of previously debilitating or deadly diseases over years, such as smallpox and polio. Blood is often drawn from patients through a needle for diagnostic purposes, administration of therapeutic treatments, monitoring levels of blood components, removing excess levels of iron or erythrocytes, or collecting blood for later uses. Although the benefits of medicinal injections and drawing blood are well known, there are some people who avoid watching or are afraid of needles being inserted into their bodies.

The present invention is a syringe cover which seeks to obscure the vision for the insertion of a needle into a patient's body, while allowing the doctor, nurse, or medical technician to view the level of fluid being injected into the patient or withdrawn from the patient. The present invention includes a collapsible sleeve which presses against the patient's body after the doctor, nurse or medical technician positions the needle for insertion. The collapsible sleeve deforms as the needle is inserted to allow the needle to be unhindered during insertion while preventing the patient from viewing the insertion of the needle.

### BRIEF DESCRIPTION OF THE DRAWINGS

FIG. 1 is a perspective view for an embodiment of the present invention, wherein the compressible sleeve includes a plurality of obscuring slats perpendicular to the mounting end.

FIG. 2 is a rear view for an embodiment of the present invention, wherein the compressible sleeve includes a plurality of obscuring slats perpendicular to the mounting end.

FIG. 3 is a front view for an embodiment of the present invention, wherein the compressible sleeve includes a plurality of obscuring slats perpendicular to the mounting end.

FIG. 4 is a perspective view for an embodiment for the present invention, wherein the compressible sleeve includes a plurality of obscuring slats angled to the mounting end.

FIG. 5 is a perspective view for an embodiment of the present invention, wherein the compressible sleeve includes a helical ribbon and a transparent transparent viewport cover across the viewport.

FIG. 6 is a perspective view for an embodiment of the present invention, wherein the patient interfacing end is discontiguous.

FIG. 7 is a plan view for an embodiment of the present invention, wherein the patient interfacing end is discontiguous, as the present invention would be cut from a sheet of material.

FIG. 8 is a top view of the present invention, wherein an adhesive layer is superimposed onto the tubular lateral wall.

FIG. 9 is a rear view for an embodiment of the present invention, wherein the present invention is positioned onto a syringe.

### DETAIL DESCRIPTIONS OF THE INVENTION

All illustrations of the drawings are for the purpose of describing selected versions of the present invention and are not intended to limit the scope of the present invention.

As some patients have an aversion or fear of needles or exhibit some anxiety upon anticipating a shot or viewing a needle, the present invention is a syringe cover for obscuring a patient's observation of needle insertions. The present invention allows doctors, nurses, medical technicians, or other medical practitioners to effectively insert a needle into a patient's body without the patient viewing the procedure. By obscuring the vision of the insertion of the needle, patients should be less likely to wince, shy away, or tense the patient's muscles during the procedure.

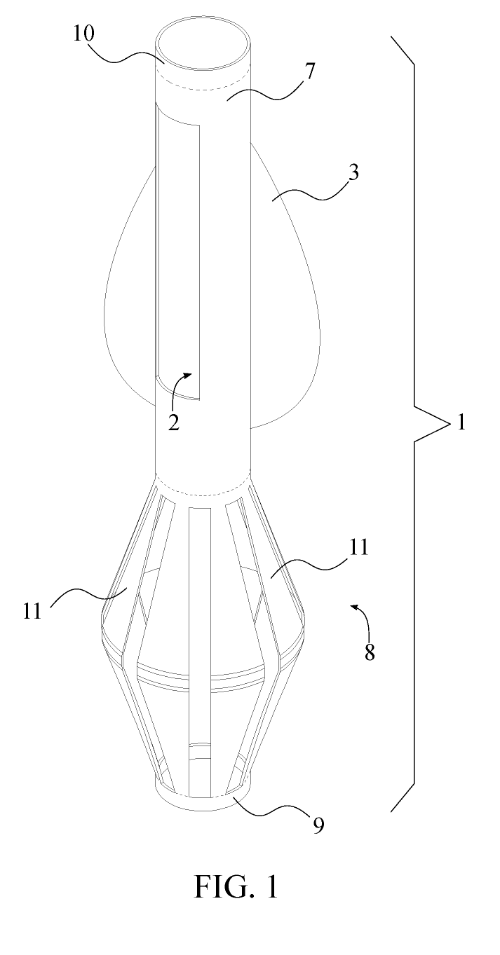

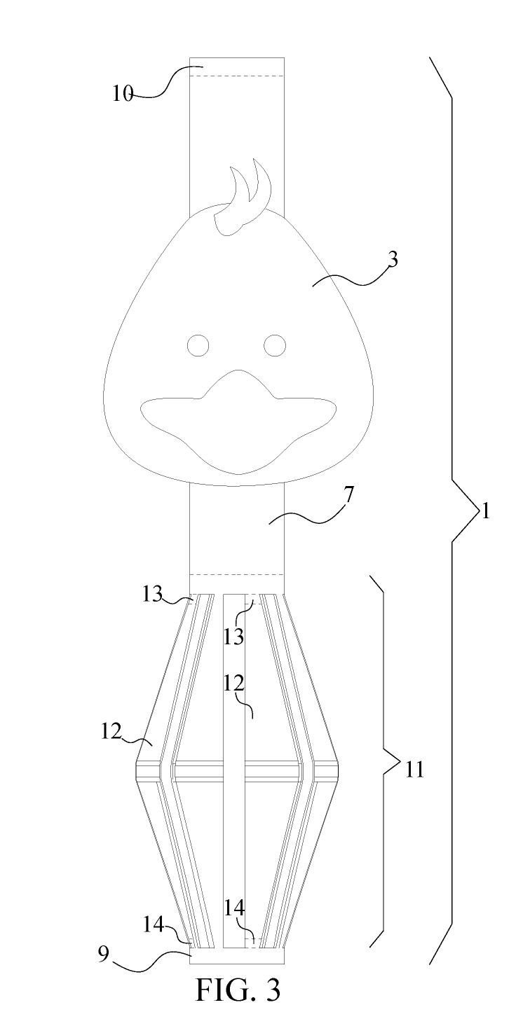

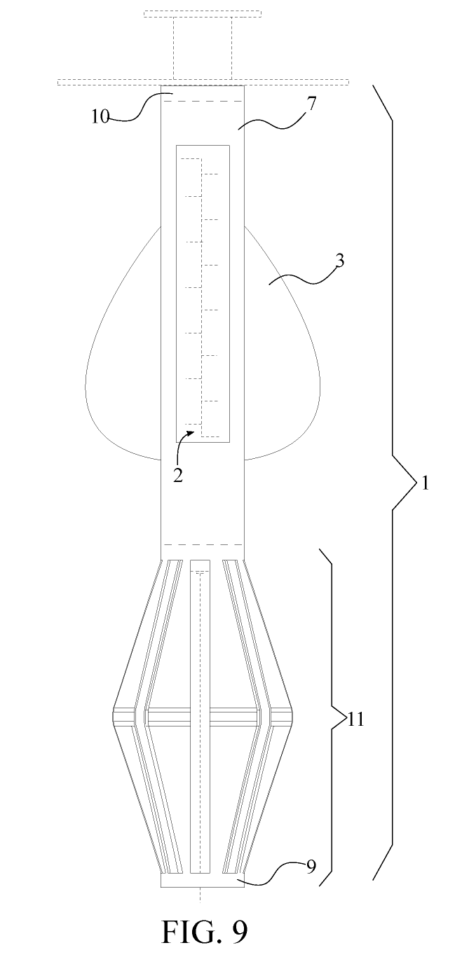

In accordance to FIG. 1 to FIG. 7 and FIG. 9, the present invention comprises a tubular sleeve 1. The tubular sleeve 1 allows the present invention to be secured about a fluid reservoir barrel of a syringe. The tubular sleeve 1 is preferred to be made of an elastic polymer. The elastic polymer allows for the tubular sleeve 1 to be fitted about the fluid reservoir barrel of the syringe. The tubular sleeve 1 comprises tubular lateral wall 7, a collapsible lateral wall 8, a patient interfacing end 9, and a flange-adjacent end 10. The tubular lateral wall 7 covers the fluid reservoir barrel in order to prevent the patient from seeing the fluid being injected into or removed from the patient's body. The collapsible lateral wall 8 allows for the ease of insertion for the needle by deforming as the needle is being inserted into the patient's skin, while obscuring the needle from the view of the patient. The collapsible lateral wall 8 is perimeterically connected to the tubular lateral wall 7. The patient-interfacing end 9 presses against the patient's body during implementation. The patient-interfacing end 9 is adjacently connected to the collapsible lateral wall 8. The patient-interfacing end 9 oppositely positioned to the tubular lateral wall 7 along the collapsible lateral wall 8, such that the patient-interfacing end 9 is oriented to press against the patient during implementation of the present invention. The patient-interfacing end 9 is preferred to be offset from a terminal end of the needle, such that the terminal end of the needle extends past the patient-interfacing end 9, as shown in FIG. 9. This configuration allows the medical practitioner to view the terminal end of the needle for insertion into the patient's body while covering a majority of the body of the needle from the patient's view. The flange-adjacent end 10 is adjacently connected to the tubular lateral wall 7. The flange-adjacent end 10 is oppositely positioned to the collapsible lateral wall 8, along the tubular lateral wall 7. In implementation, the flange-adjacent end 10 is positioned adjacent to a plunger flange, the portion of the syringe the user places their fingers to prevent the movement of the syringe while actuating a plunger of the syringe. Therefore, the tubular lateral wall 7 fully covers the syringe barrel in order to completely obscure the contents of the syringe the patients view.

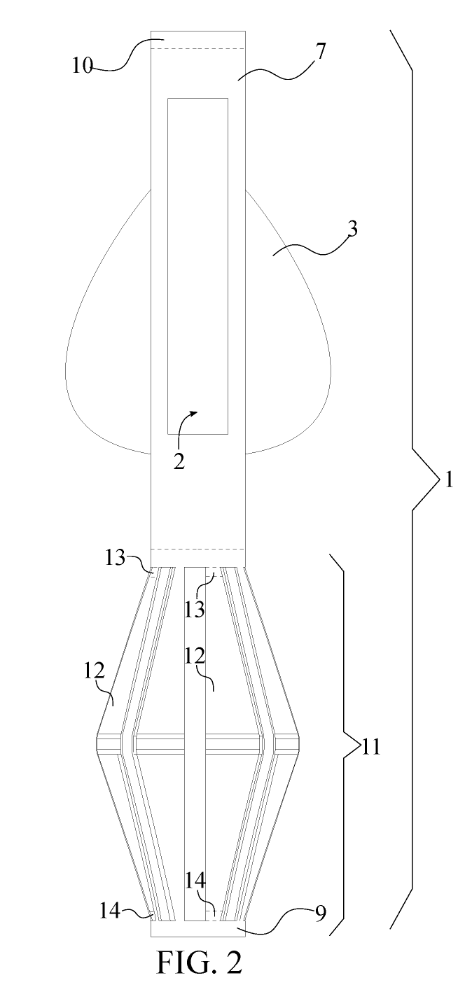

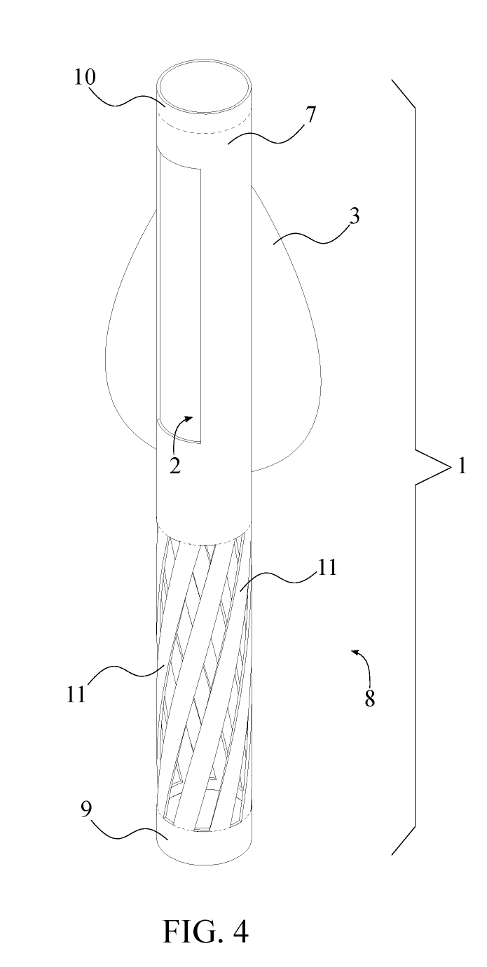

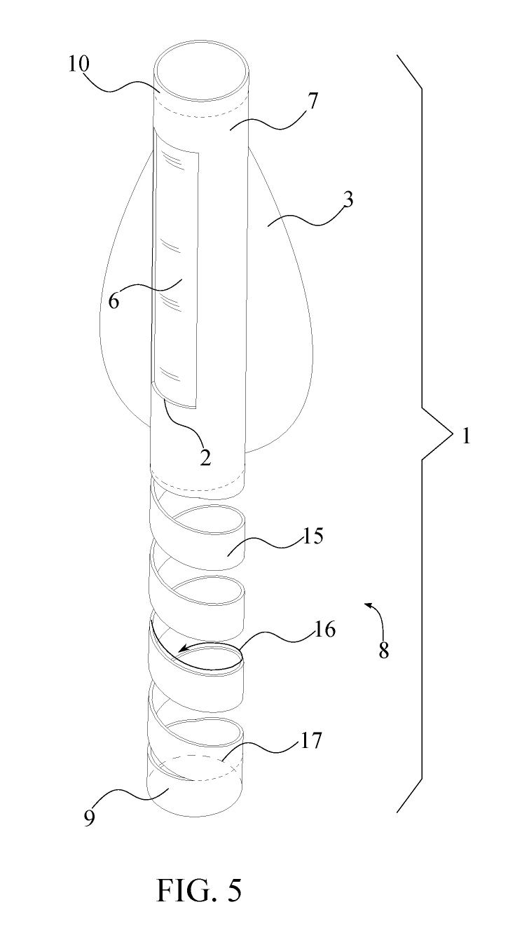

In accordance to some embodiments of the present invention, the present invention comprises a viewport opening 2, as shown in FIG. 1, FIG. 2, FIG. 4 to FIG. 6 and FIG. 9. The viewport opening 2 allows the doctor, nurse, or other medical practitioner to view the level of fluid within the fluid reservoir barrel, as shown in FIG. 9. The viewport opening 2 perpendicularly traverses through the tubular lateral wall 7 in order to allow the fluid within the fluid reservoir barrel to be visible to the medical practitioner. In some embodiments of the present invention, the present invention comprises a transparent viewport cover 6, in accordance to FIG. 5. The transparent viewport cover 6 prevents fluids from traversing through the viewport opening 2. In addition, the transparent viewport cover 6 is transparent in order to allow the doctor, nurse, or medical practitioner to view the fluid contents during implementation of the invention. The transparent viewport cover 6 is sealably integrated across the viewport opening 2 in order to prevent fluids from traversing through the viewport opening 2.

In accordance to the preferred embodiment of the present invention, the present invention comprises an aesthetic fixture 3, as detailed in FIG. 1 to FIG. 6 and FIG. 9. The aesthetic fixture 3 depicts an image which includes, but is not limited to, animals, faces, or otherwise distracting images to divert the attention of the patient from observing the insertion of a needle. The aesthetic fixture 3 is externally mounted on the tubular lateral wall 7 and is positioned between the flange-adjacent end 10 and the collapsible lateral wall 8 in order to prominently display the aesthetic fixture 3 to the patient. In some embodiments of the present invention which comprise the viewport opening 2, the aesthetic fixture 3 is opposite to the viewport opening 2 about the tubular lateral wall 7 such that the aesthetic fixture 3 does not interfere with the medical practitioner's view of the viewport opening 2 to assess the level of fluid within the fluid reservoir barrel.

In one embodiment of the present invention, the collapsible lateral wall 8 comprises a plurality of flexible obscuring slats 11, as shown in FIG. 1 to FIG. 4 and FIG. 6 to FIG. 9. The plurality of flexible obscuring slats 11 deform as the needle is inserted into a patient's body and the patient-interfacing end 9 is forced towards the tubular lateral wall 7. Each of the plurality of flexible obscuring slats 11 comprises a slat body 12, a first slat end 13, and a second slat end 14. The first slat end 13 and the second slat end 14 are positioned opposite to each other along the slat body 12. The first slat end 13 for each of the plurality of flexible obscuring slats 11 is connected to the tubular lateral wall 7. The second slat end 14 for each of the plurality of flexible obscuring slats 11 is connected to the patient-interfacing end 9. Some embodiments of the present invention for the collapsible lateral wall 8 allow the slat body 12 to deform between the first slat end 13 and the second slat end 14 to allow for the needle to be inserted unhindered by the present invention. The width of the slat body 12 is preferred to be thick enough to obscure the view of the needle from the patient's view but thin enough to allow the medical practitioner to view the depth of the needle insertion. In a perpendicular slat configuration, of the present invention, the slat body 12 is oriented perpendicular to the patient-interfacing end 9 and the tubular lateral wall 7 such that the slat body 12 bows outward at a midpoint of the slat body 12 as the patient-interfacing end 9 is forced towards the tubular lateral wall 7, in accordance to FIG. 1 to FIG. 3. In an angled slat configuration of the present invention, the slat body 12 is angled to the patient-interfacing end 9 and the tubular lateral wall 7, as shown in FIG. 4, such that the slat body 12 twists and compresses as the patient-interfacing end 9 is forced towards the tubular lateral wall 7 to obscure the needle from the patient's view.

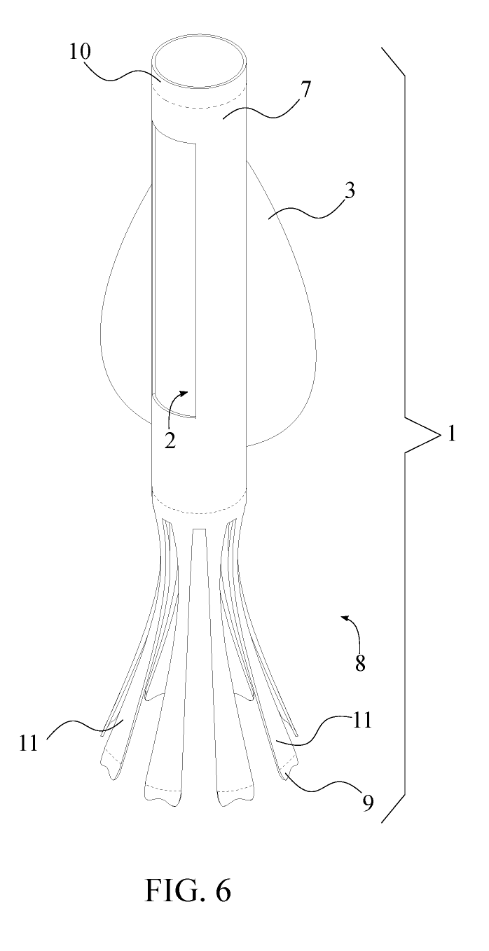

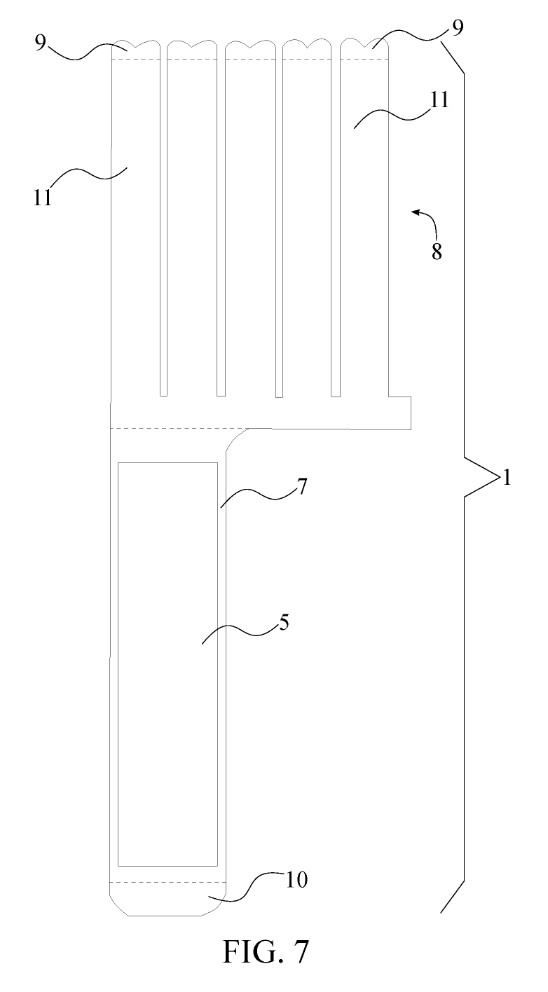

In some embodiments of the present invention, the patient-interfacing end 9 is discontiguous, in accordance to FIG. 6 and FIG. 7. This embodiment of the patient-interfacing end 9 allows the plurality of flexible obscuring slats 11 to flair outward from the syringe, as shown in FIG. 6, as the syringe is inserted into the patient's skin during implementation. With the plurality of flexible obscuring slats 11 flaring outward and the discontiguous patient interfacing end 9, the needle of the syringe is able to be fully inserted into the patient's skin. This configuration also increases the number of aesthetic deigns which the plurality of flexible obscuring slats 11 may take to provide a distraction to the insertion of the needle in a patient's skin. These aesthetic designs include, but not limited to: flower petals; animal jaws; and animal extremities, such as tentacles, legs, arms, etc.

In another embodiment of the present invention, the collapsible lateral wall 8 comprises a helical ribbon 15, as shown in FIG. 5. The helical ribbon 15 compresses as the needle is inserted into the patient's body to conceal the insertion of the needle from the patient's view. The helical ribbon 15 is connected between the patient-interfacing end 9 and the tubular lateral wall 7, such that when the patient-interfacing end 9 is forced toward the tubular lateral wall 7 the helical ribbon 15 collapses. The helical ribbon 15 compresses to be positioned concentrically within the patient-interfacing end 9 during implementation to conceal the needle. A curvature 16 of the helical ribbon 15 is preferred to be aligned coincident with a perimeter 17 of the patient-interfacing end 9, such that the curvature 16 of the helical ribbon 15 obscures the needle, although not completely, from the patient's view. The perimeter 17 is defined by the lateral shape of the patient-interfacing end 9, preferably circular.

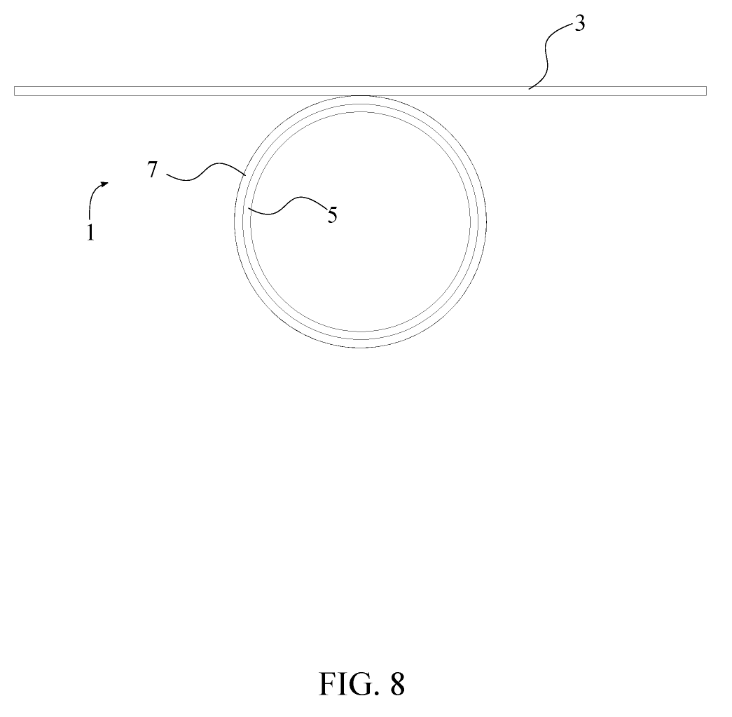

In some embodiments of the present invention, the present invention comprises an adhesive layer 5, as shown in FIG. 7 and FIG. 8. The adhesive layer 5 secures the tubular sleeve 1 to the barrel of the syringe. The adhesive layer 5 is internally superimposed onto the tubular lateral wall 7 in order to secure the tubular lateral wall 7 to the fluid reservoir barrel of the syringe. The adhesive layer 5 prevents the present invention from being dislodged from the syringe and, subsequently, the patient's view of the needle being inserted into the patient's body, the fluid being injected into the patient's body or the fluid being drawn from the patient's body. In accordance to FIG. 7, the present invention is able to be cut from a single sheet of material. The present invention is able to be wrapped around the barrel of the syringe where the adhesive layer 5 secures the present invention to the barrel of the syringe.

Although the invention has been explained in relation to its preferred embodiment, it is to be understood that many other possible modifications and variations can be made without departing from the spirit and scope of the invention as hereinafter claimed.

What is claimed is:

1. A syringe cover for obscuring a patient's observation of needle insertions comprises:
a tubular sleeve;
the tubular sleeve comprises a tubular lateral wall, a collapsible lateral wall, a patient-interfacing end, and a flange-adjacent open end;
the collapsible lateral wall being perimeterically connected to the tubular lateral wall;
the patient interfaceing end being adjacently connected to the collapsible lateral wall;
the patient interfaceing end being oppositely positioned to the tubular lateral wall along the collapsible lateral wall;
the flange-adjacent end being adjacently connected to the tubular lateral wall; and
the flange-adjacent end being oppositely positioned to the collapsible lateral wall, along to the tubular lateral end.

2. The syringe cover for obscuring a patient's observation of needle insertions, as claimed in claim 1, wherein a viewport opening perpendicularly traverses through the tubular lateral wall.

3. The syringe cover for obscuring a patient's observation of needle insertions, as claimed in claim 2, wherein a transparent viewport cover is sealably integrated across the viewport opening.

4. The syringe cover for obscuring a patient's observation of needle insertions, as claimed in claim 1, comprises:
an aesthetic fixture;
the aesthetic fixture being externally mounted on the tubular lateral wall; and
the aesthetic fixture being positioned between the flange-adjacent end and the collapsible lateral wall.

5. The syringe cover for obscuring a patient's observation of needle insertions, as claimed in claim 1, comprises:
an aesthetic fixture;
a viewport opening; and
the aesthetic fixture being opposite to the viewport opening about the tubular lateral wall.

6. The syringe cover for obscuring a patient's observation of needle insertions, as claimed in claim 1, comprises:
the collapsible lateral wall comprises a plurality of flexible obscuring slats;
each of the plurality of slats comprises a slat body, a first slat end, and a second slat end;
the first slat end and the second slat end being positioned opposite to each other along the slat body;
the first slat end for each of the plurality of flexible obscuring slats being connected to the tubular lateral wall; and
the second slat end for each of the plurality of flexible obscuring slats being connected to the patient-interfacing end.

7. The syringe cover for obscuring a patient's observation of needle insertions, as claimed in claim 6, wherein the slat body is oriented perpendicular to the patient-interfacing end and the tubular lateral wall.

8. The syringe cover for obscuring a patient's observation of needle insertions, as claimed in claim 6, wherein the slat body is angled to the patient-interfacing end and the tubular lateral wall.

9. The syringe cover for obscuring a patient's observation of needle insertions, as claimed in claim 1, comprises:
the collapsible lateral wall comprises a helical ribbon; and
the helical ribbon being connected between the patient-interfacing end and the tubular lateral wall.

10. The syringe cover for obscuring a patient's observation of needle insertions, as claimed in claim 9, wherein a curvature of the helical ribbon being aligned coincident to a perimeter of the patient-interfacing end.

11. The syringe cover for obscuring a patient's observation of needle insertions, as claimed in claim 1, wherein an adhesive layer internally superimposed onto the tubular lateral wall.
